(19) United States
(12) Patent Application Publication
(10) Pub. No.: US 2020/0269064 A1
(43) Pub. Date: Aug. 27, 2020

(54) METHOD AND APPARATUS FOR IMPROVING BLOOD FLOW IN SUBJECT'S BRAIN

(71) Applicant: LUTRONIC CORPORATION, Goyang-si (KR)

(72) Inventor: Robert Glen Calderhead, Tochigi-ken (JP)

(21) Appl. No.: 16/283,206

(22) Filed: Feb. 22, 2019

(51) Int. Cl. A61N 5/06

(52) U.S. Cl. CPC A61N 5/0618; A61N 2005/067

(57) ABSTRACT

Provided is a method of improving a blood flow in a subject's brain, the method including noninvasively sequentially delivering light energy light having a wavelength of about 810 nm to about 870 nm toward blood vessels in an occipital lobe, a temporal lobe, a frontal lobe, and a parietal lobe of the subject along a blood flow starting from one portion of a carotid artery of a subject.

### BACKGROUND

### 1. Field

One or more embodiments relate to methods and apparatuses for improving blood flow in a subject's brain.

### 2. Description of the Related Art

In the human body, the brain works by receiving oxygen and nutrients via blood flowing through a cerebral artery. When a blood vessel supplying blood to the brain is blocked or disrupted, a blocked or disrupted portion of the brain is damaged, which causes a cerebrovascular disease.

Among cerebrovascular diseases, dementia refers to a state in which a person cannot maintain his/her daily live alone due to a cognitive disorder. Dementia is caused by cognitive impairment due to a decrease in the brain cells responsible for the intellectual ability, i.e., the memory, ability to calculate, and judgement, of a patient or due to broken connections in the brain required for judgement. Dementia may cause various symptoms such as memory impairment, difficulties with language, frontal lobe disorder, spatial perception disorder, and personality changes in several stages.

A gradual decrease in the blood supply to the cerebral cortex is a major cause of dementia. To improve or recover a brain injury disease such as dementia, a smooth supply of blood to areas related to the cerebral cortex is required. For the smooth supply of blood, for example, a therapeutic method using light energy has been used.

However, the light energy applied when a treatment for the brain injury disease is used may be absorbed by various tissues such as collagen, hair follicle, and hemoglobin located in the scalp according to the wavelength characteristics of light. The absorbed light is then converted into thermal energy in tissues, e.g., brain tissues, which causes thermal damages to the brain tissues or activate particular substances and thus changes the state of the brain tissues.

Therefore, there is a need for methods and apparatuses for improving the blood flow in a subject's brain without substantially affecting the brain tissues.

### SUMMARY

One or more embodiments include a method of effectively improving a blood flow in a subject's brain without substantially affecting subject's brain tissues.

One or more embodiments include an apparatus for effectively improving blood flow in a subject's brain without substantially affecting subject's brain tissues.

Additional aspects will be set forth in part in the description which follows and, in part, will be apparent from the description, or may be learned by practice of the presented embodiments.

According to one or more embodiments, a method of improving a blood flow in a subject's brain includes noninvasively sequentially delivering light energy of light having a wavelength of about 810 nm to about 870 nm toward blood vessels in an occipital lobe, a temporal lobe, a frontal lobe, and a parietal lobe of the subject along the blood flow starting from one portion of a carotid artery of the subject.

According to one or more embodiments, an apparatus for improving a blood flow in a subject's brain includes a power supply configured to supply power to a light source, a light source configured to irradiate light having a wavelength of about 810 nm to about 870 nm, a contact device mounted with the light source and located in contact with skin at one portion of a carotid artery and a scalp at a blood vessel in an occipital plexus region, a scalp at a blood vessel in a temporal plexus region, and a scalp at a blood vessel in a parietal plexus region of a subject, and a controller configured to sequentially deliver light energy from the light source to the one portion of the carotid artery, the blood vessel in the occipital plexus region, the blood vessel in the temporal plexus region, and the blood vessel in the parietal plexus region of the subject through the contact device.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram illustrating portions of a subject's brain to which light energy is delivered and an order of delivering the light energy starting from one portion of a carotid artery to blood vessels in the brain by using a method according to an embodiment;

FIGS. 2A and 2B are schematic diagrams illustrating structures of apparatuses used to improve a blood flow in a subject's brain according to an embodiment;

FIGS. 3A to 3C are schematic side, front, and rear views of an apparatus mounted on a human skull according to an embodiment; and

FIG. 4 shows memory test results of Group A (A1, A2, A3, A4, A5, and A6) and Group B (B1, B2, B3, and B4).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, a method and an apparatus for improving blood flow in a subject's brain will be described with reference to the accompanying drawings.

It is to be understood that the terms “include” or “have” are intended to indicate the existence of elements disclosed in the specification, and are not intended to preclude the possibility that one or more other elements may exist or may be added.

Throughout the specification, the term “subject” refers to an organism including humans and animals.

As used herein, the term “carotid artery” refers to a blood vessel that carries blood from the heart to the brain to supply energy thereto to enable the brain to function smoothly and includes external carotid artery and internal carotid artery.

In this specification, the term “blood vessel” is intended to include all of arteries, veins, and capillaries.

The expression “noninvasively delivering light energy sequentially to blood vessels” used herein refers to sequentially delivering light energy to a blood vessel located in a first component, a blood vessel located in a second component, a blood vessel located in a third component, and a blood vessel located in a fourth component by irradiation without using a surgical operation, or the like. The first component may be an occipital lobe. The second component may be a temporal lobe. The third component may be a frontal lobe. The fourth component may be a parietal lobe.

As used herein, the term “dispose” is intended to include both direct and indirect contacts with a target region.

A method of improving a blood flow in a subject's brain according to an embodiment includes noninvasively sequentially delivering light energy of light having a wavelength of about 810 nm to about 870 nm toward blood vessels in an occipital lobe, a temporal lobe, a frontal lobe, and a parietal lobe of the subject along a blood flow by starting from one portion of a carotid artery of the subject.

The method of improving the blood flow in the subject's brain may effectively increase blood flow in the brain by delivering light energy to a cerebral cortex area of the subject, i.e., blood vessels located in the occipital lobe, the temporal lobe, the frontal lobe, and the parietal lobe, sequentially starting from the carotid artery in the same order of supplying cerebral blood flow. The cerebral cortex includes a large number of neurons located on the surface of a cerebrum and plays key roles in memory, concentration, thought, language, awareness, consciousness, and the like. To prevent damages to the cerebral cortex area responsible for such key roles or to treat the damages, the blood flow in a subject's brain may be effectively improved by sequentially delivering light energy to the cerebral cortex areas as targets. Furthermore, the method of improving the blood flow in a subject's brain may improve cognitive ability or recover brain injury.

The wavelength of the light energy may be from about 810 nm to about 870 nm. For example, the wavelength of the light energy may be from about 820 nm to about 860 nm, for example, from about 820 nm to about 850 nm, for example, from about 820 nm to about 840 nm, for example, about 830 nm. The wavelength of the light energy is a wavelength of near infrared light having an optical density (OD, log units) of about 3.2 or less. Near infrared light may sufficiently be delivered from the scalp deeply to blood vessels in a neural network of the cerebral cortex area. In addition, since the light energy is converted into thermal energy, the blood flow in the subject's brain may be improved without any damage to brain tissues.

The light energy may be of a coherent or non-coherent type. As a light source of the light energy, a laser diode (LD) or a light emitting diode (LED) may be used. When the light energy is of a coherent type, the light energy may be intensively delivered to the subject as a high energy. When the light energy is of a non-coherent type, the light energy may be delivered to wide blood vessel areas in the subject's brain by using a long wavelength.

The light energy may be of a pulse-type. A continuous-type light energy may consume more power than the pulse-type light energy and may cause damages to the brain tissues when more heat than required is applied thereto. The pulse-type light energy is delivered deep into the blood vessels in the subject's brain by adjusting interpulse intervals and using high-peak power at the same frequency as the continuous-type light energy without separately adjusting temperature applied to the brain tissues. Thus, the pulse-type energy may effectively increase blood flow in the subject's brain. The pulse-type energy may be effective when the light energy is of a coherent type.

The light energy may have a square-wave pattern. The square-wave pattern may increase blood flow more efficiently in the subject's brain by causing beneficial resonance in brain cells and tissues of the subject by modifying inherent frequencies of the human body.

According to the method of the present embodiment, the noninvasively sequentially delivering light energy toward the blood vessels may include disposing light sources on the skin of the subject at one portion of the carotid artery and on the scalp at a blood vessel in an occipital plexus region, on the scalp at a blood vessel in a temporal plexus region, and on the scalp at a blood vessel in a parietal plexus region, and sequentially delivering light energy from the light sources to the portion of the carotid artery, the blood vessel in the occipital plexus region, the blood vessel in the temporal plexus region, and the blood vessel in the parietal plexus region of the subject.

The noninvasively sequentially delivering of the light energy to the blood vessels may be performed by disposing the light sources on the skin of the subject at one portion of the carotid artery, for example, one portion of an internal carotid artery, and on the scalp at the blood vessel in the occipital plexus, on the scalp at the blood vessel in the temporal plexus, and on the scalp at the blood vessel in the parietal plexus. In this case, the light sources may be disposed on the skin and the scalp in direct contact therewith or mounted on and/or in a device or apparatus in contact with the skin and the scalp. The device or apparatus may be manufactured using a material stretching to have a shape corresponding to a shape of a subject's skull and transmitting light energy. Thus, light energy may be delivered from the light source sequentially to the one portion of the carotid artery, the blood vessel in the occipital plexus region, the blood vessel in the temporal plexus region, and the blood vessel in the parietal plexus region of the subject.

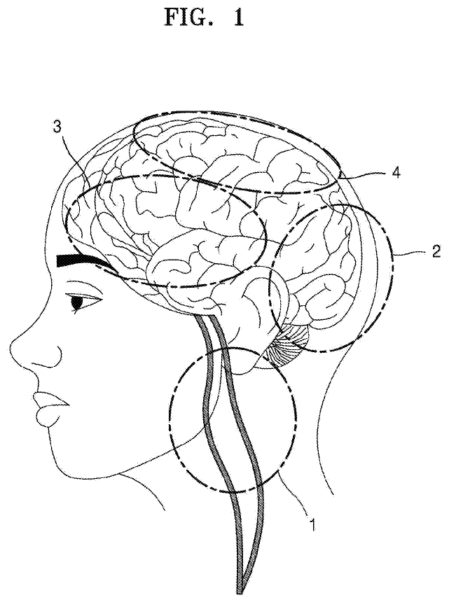

FIG. 1 is a schematic diagram illustrating portions of a subject's brain to which light energy is delivered and an order of delivering the light energy to blood vessels in the brain starting from one portion of a carotid artery by using a method according to an embodiment.

FIG. 1 shows that the light energy is delivered sequentially to a blood vessel in an occipital plexus region 2, a blood vessel in a temporal plexus region 3, and a blood vessel in a parietal plexus region 4 of a subject in the order of cerebral blood supply starting from one portion of a carotid artery 1 according to the method. In this case, the occipital plexus region 2 is a neural network region for processing visual information and analyzes location, shape, and motion state of an object based on visual information obtained by eyes. The temporal plexus region 3 is a neural network region for processing auditory information, emotion, realistic memory, and visual memory information. Damages to the temporal plexus region 3 may cause impairment in non-verbal auditory stimulation, language formation, memory, and cognitive ability. The parietal plexus region 4 has a somatosensory cortex and a sensory association area, is involved in somatosensory processing such as tactile, pressure, and pain, and is responsible for sensory signals from skin, musculoskeletal system, internal organs, and taste buds. Since the light energy is sequentially delivered to blood vessels in the brain in the order of the cerebral blood supply according to the method, blood flow may be efficiently increased in the brain. Thus, impairment of visual, auditory, language, memory, cognitive ability, or sensory abilities of the occipital plexus region 2, the temporal plexus region 3, and the parietal plexus region 4 may be prevented or treated, in case of occurrence.

According to the method, the light energy may be sequentially delivered from the light sources to the one portion of the carotid artery, the blood vessel in the occipital plexus region, the blood vessel in the temporal plexus region, and the blood vessel in the parietal plexus region of the subject at least one cycle for a duration of about 5 seconds to about 15 seconds per cycle. The light energy may be delivered sequentially to the one portion of the carotid artery and the blood vessels in the plexus regions of the subject for a duration of, for example, about 6 seconds to about 14 seconds, for example, about 7 seconds to about 13 seconds, for example, about 8 seconds to about 12 seconds, for example, about 9 seconds to about 11 seconds, and for example, about 10 seconds. After the light energy is delivered to the one portion of the carotid artery and the blood vessels in the plexus regions of the subject, the delivering of light energy sequentially to the one portion of the carotid artery, the blood vessel in the occipital plexus region, the blood vessel in the temporal plexus region, and the blood vessel in the parietal plexus region of the subject may be performed two cycles, three cycles, or more. The cycles may be repeated to the extent that the brain tissues are not substantially damaged by an increase in temperature. The light source may be an LED or an LD. For example, when an LED light source is used as the light source, the light energy is delivered less intensively to a larger area of blood vessels in the brain when compared with the case of using an LD light source. Thus, the number of cycles described above may be increased.

When the light source is an LD light source, the method may further include cooling the regions where the light energy was delivered sequentially to the blood vessels from the light source.

The light energy may be applied, for example, at an energy density of about 5 J/cm² or less per cycle. For example, the light energy may be delivered to the one portion of the carotid artery and blood vessels in the plexus regions of the subject at an energy density of about 5 J/cm² or less for a duration of about 5 seconds to about 15 seconds per cycle.

The light energy may be applied, for example, at a power density of about 500 mW/cm² or less per cycle. For example, the light energy may be applied to the one portion of the carotid artery and blood vessels in the plexus regions of the subject at an energy density of about 5 J/cm² or less and a power density of about 500 mW/cm² or less for a duration of about 5 seconds to about 15 seconds per cycle.

The light energy may be applied, for example, at a peak power of about 5 to about 15 W per cycle. For example, the light energy may be applied to the one portion of the carotid artery and blood vessels in the plexus regions of the subject at an energy density of about 5 J/cm² or less, a power density of about 500 mW/cm² or less, and a peak power of about 5 to about 15 W for a duration of about 5 seconds to about 15 seconds per cycle.

The above-described duration, energy density, power density, and peak power of the light energy per cycle may be effectively applied when the light source is an LD. In addition, the duration, energy density, power density, and peak power of the light energy per cycle may be appropriately adjusted within the ranges described above according to a health state of the subject to which the light energy is delivered. Also, when the light source is an LD, the duration, energy density, power density, and peak power of the light energy per cycle are not limited thereto and appropriately modified or changed. In addition, a session for delivering the light energy to the subject's brain is not particularly limited.

The method may be used to treat a subject's brain injury by improving the blood flow in the brain. The subject's brain injury refers to an abnormality caused by an insufficient supply of blood to the cerebral cortex, which results in damages or injuries to brain cells. For example, the subject's brain injury may include a stroke, cerebral infarction, cerebral hemorrhage, dizziness, senile dementia, post-stroke dementia, or post-traumatic brain injury.

The method may be used to treat diseases associated with a gradual decline in blood supply to the cerebral cortex, such as senile dementia, post-stroke dementia, or post-traumatic brain injury. Symptoms caused by a gradual decrease in blood supply to the cerebral cortex may progress in three stages including a first stage with poor recent memory, decreased ability to deduce, calculate, and learn new things, visual-spatial confusion and disorientation, and lack of insight or judgment; a second state with poor cognitive abilities such as learning and judgement, emotional instability, and getting angry easily; and a third state with loss of all cognitive abilities, difficulty in daily living such as eating and bathing alone, decrease in personal hygiene, and weight loss. In order to reduce or recover these symptoms progressing in several stages, there is a need to smoothly deliver blood, which supplies oxygen, nutrients, and electrolytes, to blood vessels in the brain. The method may be used to more efficiently treat diseases related in gradual decline of blood supply by delivering light energy to the blood vessels in the brain sequentially in the order of cerebral blood supply.

According to the method, short-term memory may be improved by supplying oxygen, nutrients, and electrolytes to the brain by improving blood flow in blood vessels in damaged areas of the brain, e.g., the cerebral cortex.

The method may aid study of a subject by improving blood flow in the brain. For example, when the subject is a student, the method may improve learning ability by improving short-term memory and cognitive ability.

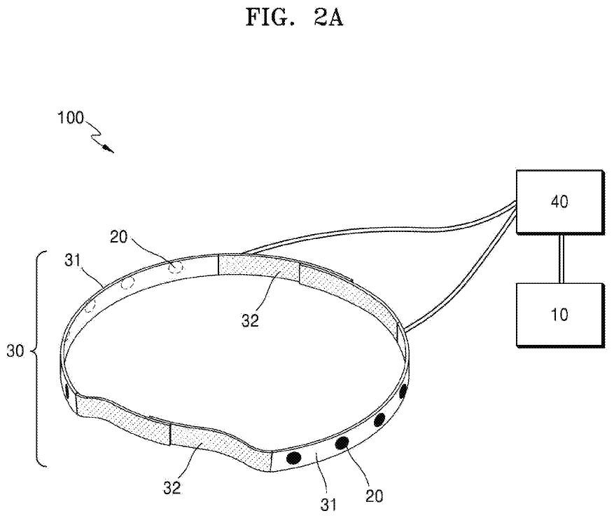

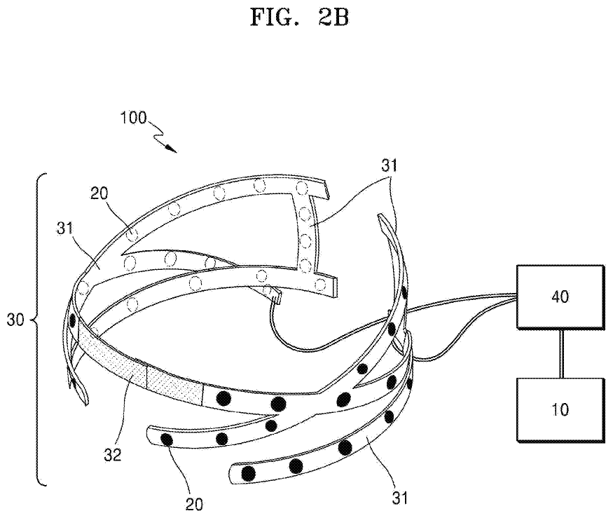

FIGS. 2A and 2B are schematic diagrams illustrating structures of apparatuses used to improve blood flow in brains according to an embodiment.

Referring to FIGS. 2A and 2B, an apparatus for improving blood flow in a subject's brain 100 according to an embodiment may include a power supply 10, a light source 20, a contact device 30 on which the light source 20 is mounted, and a controller 40. The contact device 30 on which the light source 20 is mounted may be manufactured in various shapes according to a shape of a skull.

The apparatus for improving blood flow in the subject's brain 100 may include the power supply 10 configured to supply power to the light source 20, the light source configured to deliver light energy having a wavelength of about 810 nm to about 870 nm, the contact device 30 mounted with the light source 20 and located in contact with the skin at one portion of a carotid artery and the scalp at a blood vessel in an occipital plexus region, the scalp at a blood vessel in a temporal plexus region, and the scalp at a blood vessel in a parietal plexus region of a subject, and the controller 40 configured to deliver light energy from the light source 20 sequentially to the one portion of the carotid artery, the blood vessel in the occipital plexus region, the blood vessel in the temporal plexus region, and the blood vessel in the parietal plexus region of the subject through the contact device 30.

The power supply 10 may be a mobile power supply or a desktop power supply.

The light source 20 may be an LED or an LD.

The contact device 30 may include a plurality of contact units including a polymer strip 31 transmitting light energy and a physically adhesive strap 32. The light source 20 may be mounted on the surface and/or inside of the polymer strip 31 of the contact device 30 transmitting light energy in a predetermined interval.

The polymer strip 31 transmitting light energy may be stretchable. A material used to manufacture the stretchable polymer strip 31 transmitting light energy may include, for example, polyamide, polyvinyl chloride, polyethylene, polymethyl methacrylic acid, polyurethane, polyethylene terephthalate, polyolefin, or a copolymer thereof. The physically adhesive strap 32 may be in the form of Velcro strips, hooks, straps, buttons, or snaps such that the polymer strip 31 transmitting the light energy is maintained in close contact with the scalp of the subject's skull.

The contact device 30 may include a plurality of contact units in which a polymer strip mounted with the light source 20 and transmitting light energy is in close contact with the scalp at positions corresponding to the one portion of the carotid artery, the blood vessel in the occipital plexus region, the blood vessel in the temporal plexus region, and the blood vessel in the parietal plexus region of the subject by using the adhesive strap.

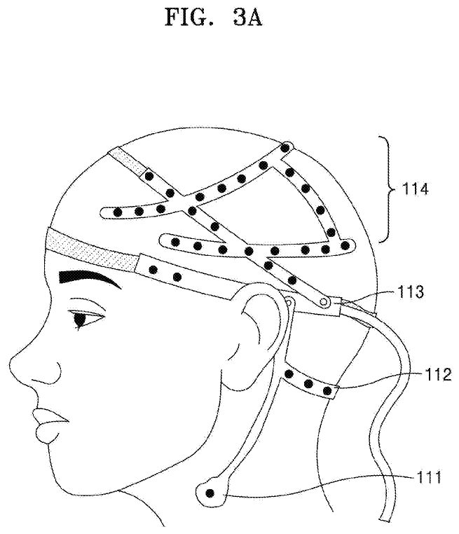

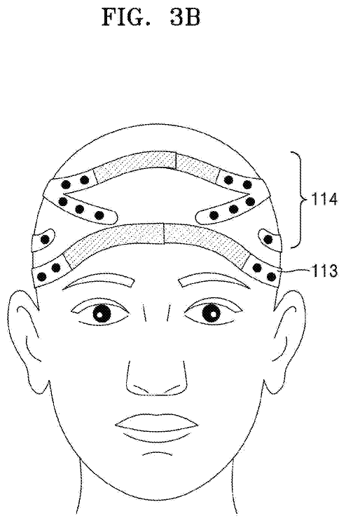

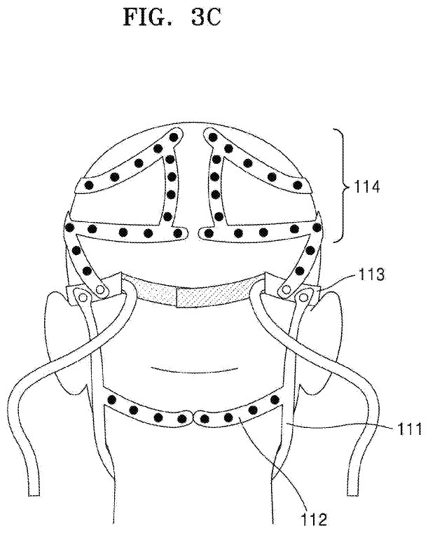

FIGS. 3A to 3C are schematic side, front, and rear views of an apparatus according to an embodiment mounted on a human skull.

Referring to FIGS. 3A to 3C, a side view, a front view, and a rear view of the apparatus including the contact device 30 including a plurality of contact units, provided with the light sources 20, and mounted on the human skull according to an embodiment are shown and cables to be connected to the power supply and/or the controller are schematically shown.

The plurality of contact units may include a first contact unit 111 surrounding the skin where the one portion of the carotid artery of the subject is located, a second contact unit 112 surrounding the scalp of the occipital lobe where the blood vessel in the occipital plexus region of the subject is located, a third contact unit 113 surrounding the scalp of the temporal lobe where the blood vessel in the temporal plexus region of the subject is located, and a fourth contact unit 114 surrounding the scalp from the frontal lobe or the temporal lobe to the scalp of the parietal lobe where the blood vessel in the parietal plexus region is located. The first contact unit 111, the second contact unit 112, the third contact unit 113, and the fourth contact unit 114 are electrically connected with each other. The light energy may be delivered sequentially to the first contact unit 111, the second contact unit 112, the third contact unit 113, and the fourth contact unit 114 from respective light sources thereof at least one cycle. In this case, the first contact unit 111, the second contact unit 112, the third contact unit 113, and the fourth contact unit 114 may be each independently units including a plurality of sub-contact units. The plurality of sub-contact units may be disposed in one direction or various directions along blood vessels in a neural network in the brain.

Although not shown in the drawings, the apparatus for improving blood flow in the subject's brain may further include a sensor configured to measure temperatures of the one portion of the carotid artery, the blood vessel in the occipital plexus region, the blood vessel in the temporal plexus region, and the blood vessel in the parietal plexus region of the subject as needed.

The apparatus for improving blood flow in the subject's brain may further include a cooler configured to cool the region where the light energy was delivered when the temperature is increased by the delivered light energy to a temperature higher than that causing damages to brain tissues.

According to the method and the apparatus for improving blood flow in the subject's brain according to an embodiment, the blood flow may effectively be improved in the subject's brain with no substantial reactions with carotid artery tissues by delivering light energy sequentially to the blood vessels along the blood flow starting from one portion of the carotid artery.

Hereinafter, the present disclosure will be described in more detail according to the following examples. However, the following examples are merely presented to exemplify the present disclosure, and the scope of the present disclosure is not limited thereto.

### Experimental Example 1: Profile of Moods Scale (POMS) Test

10 subjects in total were classified into Group A (6 subjects) and Group B (4 subjects). After treatment with laser diodes (LDs) and sham treatment lasting for 5 weeks, with two sessions being held each week, separated by at least one day, the subjects scored mood-related items twice: once at baseline, and at the end of the final session. The test results are shown in Table 1 below.

The 6 subjects in the actual treatment Group A were treated with laser diodes (LDs) having a wavelength of 830 nm for about 10 seconds, at an energy density of about 5 J/cm², and at a power density of about 500 mW/cm² starting from one portion of the carotid artery sequentially to blood vessels in the brain as illustrated in FIG. 1 to deliver the energy of the LDs thereto. The 4 subjects in the sham treatment Group B were treated with LDs identical to those used for the treatment group, which to all intents and purposes were delivering the same parameters and were applied to the subjects in exactly the same protocol as the treatment group, with the exception that no pumping energy was being delivered to the LDs from the control apparatus.

In the POMS test, the subjects scored a number of mood-related items from zero (none) to 4 (strong feeling) in the negative categories of tension, depression, anger, fatigue, and confusion verses the positive category of vigor. An overall POMS score was obtained by subtracting an average score of the positive items of each group from an average total score of the negative items of each group.

TABLE 1

| | Average scores | | Overall POMS |
|---|---|---|---|
| Group | Negative items | Positive items | (tPOMS) |
| A | 74 | 83 | −9 |
| B | 51 | 17 | 34 |

Referring to Table 1, Group A had a lower overall POMS (tPOMS) than Group B. Therefore, it may be confirmed that Group A had a more positive feeling than Group B.

### Experimental Example 2: Memory Test

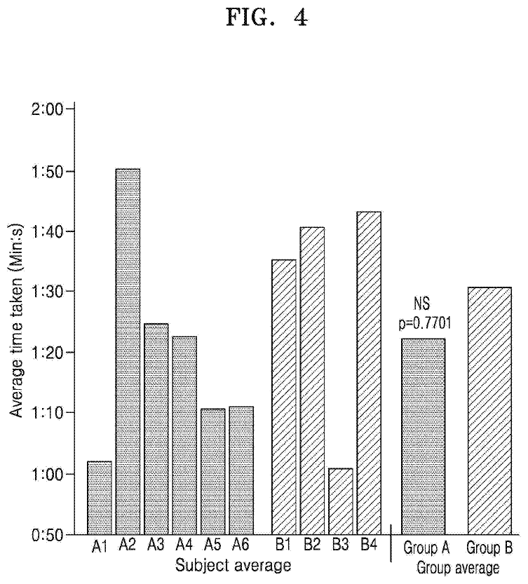

The 10 subjects in total, 6 subjects in Group A of Experimental Example 1 and 4 subjects in Group B were subjected to a memory test. As the memory test, a playing card-matching memory test was used with the time taken to complete the test being recorded for each session. An increasing number of cards was added every second weeks. The results are shown in FIG. 4.

Referring to FIG. 4, Group A showed a shorter average time taken to complete the playing-cased matching memory test than Group B. Thus, it may be confirmed that the light treatment effect on improvement of memory was demonstrated in Group A in comparison with Group B.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A method of improving a blood flow in a subject's brain, the method comprising:
noninvasively sequentially delivering light energy of light having a wavelength of about 810 nm to about 870 nm toward blood vessels in an occipital lobe, a temporal lobe, a frontal lobe, and a parietal lobe of a subject along a blood flow starting from one portion of a carotid artery of the subject.

2. The method of claim 1, wherein the light energy is of a coherent type or a non-coherent type.

3. The method of claim 1, wherein the light energy is of a pulse-type.

4. The method of claim 1, wherein the light energy has a square-wave pattern.

5. The method of claim 1, wherein the noninvasively sequentially delivering of the light energy to blood vessels comprises:
disposing light sources on subject's skin at the one portion of the carotid artery and a scalp at a blood vessel in an occipital plexus region, a scalp at a blood vessel in a temporal plexus region, and a scalp at a blood vessel in a parietal plexus region of the subject; and
sequentially delivering the light energy from the light sources to the one portion of the carotid artery, the blood vessel in the occipital plexus region, the blood vessel in the temporal plexus region, and the blood vessel in the parietal plexus region of the subject.

6. The method of claim 5, wherein the light energy is sequentially delivered from the light sources at least one cycle to the one portion of the carotid artery, the blood vessel in the occipital plexus region, the blood vessel in the temporal plexus region, and the blood vessel in the parietal plexus region of the subject for a duration of about 5 seconds to about 15 seconds per cycle.

7. The method of claim 5, wherein the light source comprises a light emitting diode (LED) or a laser diode (LD).

8. The method of claim 5, further comprising:
cooling regions where the light energy was delivered sequentially from the light sources to the blood vessels when the light sources are LDs.

9. The method of claim 1, wherein the light energy is applied at an energy density of about 5 J/cm² or less per cycle.

10. The method of claim 1, wherein the light energy is applied at a power density of about 500 mW/cm² or less per cycle.

11. The method of claim 1, wherein the light energy is applied at a peak power of about 5 W to about 15 W per cycle.

12. The method of claim 1, wherein the method is used to treat a subject's brain injury by improving the blood flow in the brain.

13. The method of claim 12, wherein the subject's brain injury comprises a stroke, cerebral infarction, cerebral hemorrhage, dizziness, senile dementia, post-stroke dementia, or post-traumatic brain injury.

14. The method of claim 1, wherein the method is used to aid study of the subject by improving the blood flow in the brain.

15. An apparatus for improving a blood flow in a subject's brain, the apparatus comprising:
a power supply configured to supply power to a light source;
a light source configured to irradiate light energy of light having a wavelength of about 810 nm to about 870 nm;
a contact device mounted with the light source and located in contact with subject's skin at one portion of a carotid artery and a scalp at a blood vessel in an occipital plexus, a scalp at a blood vessel in a temporal plexus, and a scalp at a blood vessel in a parietal plexus of a subject; and
a controller configured to deliver light energy from the light source sequentially to the one portion of the carotid artery, the blood vessel in the occipital plexus region, the blood vessel in the temporal plexus region, and the blood vessel in the parietal plexus region of the subject through the contact device.

16. The apparatus of claim 15, wherein the power supply comprises a mobile power supply or a desktop power supply.

17. The apparatus of claim 15, wherein the light source comprises a light emitting diode (LED) or a laser diode (LD).

18. The apparatus of claim 15, wherein the contact device comprises a plurality of contact units comprising a polymer strip transmitting the light energy and a physically adhesive strap.

19. The apparatus of claim 15, wherein the plurality of contact units comprises:
a first contact unit surrounding the skin where the one portion of the carotid artery of the subject is located;
a second contact unit surrounding the scalp of an occipital lobe where the blood vessel in the occipital plexus region of the subject is located;
a third contact unit surrounding the scalp of a temporal lobe where the blood vessel in the temporal plexus region of the subject is located; and
a fourth contact unit surrounding the scalp from a frontal lobe or the temporal lobe of the subject to a parietal lobe where the blood vessel in the parietal plexus region is located,
wherein the first contact unit, the second contact unit, the third contact unit, and the fourth contact unit are electrically connected with each other, and
the light energy is delivered respectively sequentially from the light sources toward the first contact unit, the second contact unit, the third contact unit, and the fourth contact unit at least one cycle.

20. The apparatus of claim 15, further comprising:
a sensor configured to measure temperatures of the one portion of the carotid artery, the blood vessel in the occipital plexus region, the blood vessel in the temporal plexus region, and the blood vessel in the parietal plexus region of the subject.
